(19) United States
(12) Patent Application Publication
(10) Pub. No.: US 2017/0165494 A1
(43) Pub. Date: Jun. 15, 2017

(54) Feedthrough Of A Medical Electronic Device, Method For Producing Same, And Medical Electronic Device

(71) Applicant: BIOTRONIK SE & Co. KG, Berlin (DE)

(72) Inventors: Daniel Kronmueller, Nuernberg (DE); Stefan Eck, Hoechstadt (DE)

(21) Appl. No.: 15/359,890

(22) Filed: Nov. 23, 2016

(30) Foreign Application Priority Data
Dec. 15, 2015 (DE) 10 2015 121 818.6

(51) Int. Cl. A61N 1/375; H01B 17/26; A61N 1/39; H05K 5/02; A61N 1/36; A61N 1/362

(52) U.S. Cl. CPC A61N 1/3754; A61N 1/36032; A61N 1/362; A61N 1/3956; H05K 5/0247; H01B 17/265

(57) ABSTRACT

A feedthrough of a medical electronic device, which in particular is implantable and has a device housing in which electronic and/or electrical function units are housed and which has a housing opening closed by the feedthrough, wherein the feedthrough has an insulating body, a feedthrough flange surrounding the insulating body and fixed to the housing opening, and at least one connection element penetrating through the insulating body for the external connection of at least one component of the device, wherein the connection element or at least one connection element consists at least in part, in particular substantially fully, of a shape-memory alloy.

### CROSS-REFERENCE TO RELATED APPLICATIONS

This patent application claims the benefit of and priority to co pending German Patent Application No. DE 10 2015 121 818.6, filed on Dec. 15, 2015 in the German Patent Office, which is hereby incorporated by reference in its entirety.

### TECHNICAL FIELD

The present invention relates to a feedthrough of an implantable medical electronic device and also to a device of this type. This device typically comprises a device housing, in which electronic and electrical function units are housed. A feedthrough of this type comprises an insulating body, particularly made of ceramic or glass, a feedthrough flange surrounding the insulating body, and at least one connection element penetrating through the insulating body for the external connection of an electrical or electronic component of the device. The present invention also relates to a medical electronic device, and to a method for producing same. Furthermore, the present invention relates to a plug part of a medical electronic modular unit, which plug part has an insulating body and at least one connection element penetrating through the insulating body for the external connection of an electrical line of the modular unit, and also to a corresponding modular unit and a method for producing same.

### BACKGROUND

Implantable devices of the above-mentioned type have long been used on a mass scale, in particular as cardiac pacemakers, implantable cardioverters (especially defibrillators), or also as cochlear implants, for example. However, said device may also be a less complex device, such as an electrode or sensor line. Medical electronic modular units within the sense of the embodiments hereinafter are, for example, electrode lines for use with cardiac pacemakers or implantable defibrillators, nerve and brain stimulators, sensor lines, or the like.

Most implantable medical electronic devices of practical significance are intended to deliver electrical pulses to excitable body tissue via suitably placed electrodes. Many devices can also selectively measure signals of the nerve tissue in the patient's body and can record or evaluate said signals over a relatively long period of time in order to select individually tailored therapy and in order to monitor the success of the treatment in vivo.

In order to perform this function, electronic/electrical function units for generating and regulating the pulses and for measuring stimuli are housed in the housing of the device. Electrodes or connections are provided externally on the device for at least one electrode line, in the distal end portion of which the electrodes are attached to the tissue for pulse transmission.

For this purpose, an electrical connection must be established between the electrical and/or electronic components arranged in the housing interior and the respective electrode lines. This electrical connection is generally provided by means of a feedthrough and/or what is known as a header. Here, a feedthrough of this type ensures at least one electrical connection between the interior of the housing and the exterior, and at the same time hermetically seals off the housing of the implant. The header, fastened via the feedthrough, guides the electrical connection of the feedthrough further to a contact point and serves for plugging the at least one electrode line into a corresponding, usually standardized socket. An electrical contact is thus produced between the implant and the connection piece of the electrode line at the contact points of the socket. A feedthrough and a header can also be provided in a single component. In this case as well, a combined component of this type will be referred to hereinafter generally as a feedthrough.

In particular, feedthroughs which are joined from the various components by means of a hard-soldering process (brazing process) are widespread.

The insulating body of the feedthrough consists substantially of ceramic or glass. The flange, which is required in order to hermetically seal the housing or implant with the feedthrough, usually consists of a metal (for example, titanium) or an alloy (for example, Ti-6Al-4V). It is considered to be advantageous to produce flange material and housing or implant material from materials of the same type so as to be able to easily join these to one another. The flange of the feedthrough is usually welded to the housing.

The contact elements penetrate through the insulating body and are electrically insulated from one another and with respect to the flange. They usually consist of highly conductive metals (e.g., tantalum, niobium, titanium, platinum) or alloys (e.g., PtIr, FeNi, 316L). Wire portions or what are known as pins are frequently used for the production of contact elements. Details regarding the production of assembled, soft-solderable contact elements and variants thereof are disclosed for example in European Patent No. EP 2 371 418 A2.

Known feedthroughs of this type largely meet the requirements placed thereon in terms of gas tightness and biocompatibility.

It is also known, in the case of feedthroughs or headers of medical electronic devices, to provide inserts made of shape-memory material, as is taught, for example, in United States Publication No. 2002/0165588 or United States Publication No. 2014/0161973.

The connection or contact elements must be arranged in defined positions relative to the feedthrough flange so that they can be connected to the other components (e.g., circuit board, header) of the medical implant in subsequent processes (e.g., welding, soft-soldering, crimping) following the production of the feedthrough. An excessive deviation of the position of the contact elements means that the component cannot be processed. For example, in the case of multi-pole feedthroughs, an addition of tolerances may mean that not all contact points lie above the counter contact or that the connection elements do not meet in one plane.

In order to ensure the position in the further-processing process, the following measures can be taken:

determining narrow position or manufacturing tolerances of the contact elements during production, transport and further processing of the feedthrough,

special packaging for protecting the pins of the feedthroughs,

manual finishing of the pins (bending, positioning) prior to the further processing, and/or

screening inspections prior to the further processing.

The observance of the position and manufacturing tolerances of the contact elements relative to the flange over the entire production process constitutes a great challenge in terms of manufacture and logistics. If the tolerances are very narrow, the feedthrough must be handled during the production sequence by means of specially produced manufacturing means and transport containers. Packaging and transport containers of the feedthroughs must additionally be designed such that the position and tolerances of the contact elements relative to the flange are not adversely affected by the handling or by vibration and storage conditions. The risk of rejection rises with the number of pins and the number of handling and storage processes. In order to achieve a high yield, additional process and inspection steps must be integrated. In order to ensure that only defect-free feedthroughs reach the subsequent assembly process, screening inspections and additional finishing steps must be integrated in the production sequence. The additional effort during the production of the feedthrough must be factored in and thus increases the cost of the feedthrough and the medical implant.

The present invention is directed toward overcoming one or more of the above-mentioned problems.

### SUMMARY

An object of the present invention is therefore to provide an improved feedthrough of a medical electronic device and an improved plug part of a medical electronic modular unit, which, with regard to their manufacture, require a lesser inspection and handling effort in respect of the final assembly of the device and modular unit respectively and thus lead to a reduction of the product costs. A suitable production method of a corresponding device and modular unit will also be specified.

At least this object is achieved in terms of its device aspects by a feedthrough having the features of claim 1 and by a medical electronic device having the features of claim 9, and in accordance with a relatively independent aspect of the present invention by a plug part having the features of claim 12, and a medical electronic modular unit having the features of claim 14. In terms of its method aspects, at least the object is achieved by methods having the features of claim 10 and claim 15. Expedient developments are disclosed in the corresponding dependent claims.

One concept of the present invention is to present a way of producing hermetically sealed plug connectors based on metal-ceramic composite materials. The present invention also includes the concept of utilizing the known shape-memory effect in the context of medical electronic devices or modular units in order to compensate for or reverse manufacturing-induced position errors or position shifts or dimensional changes of essential parts, in particular of the contact elements with respect to the flange, caused by handling processes prior to the final assembly. The present invention also includes the concept of applying this notion especially to at least some of the connection elements of the feedthrough of a medical electronic device or of the plug part of a medical electronic modular unit. Lastly, provision is made in accordance with the present invention so that the connection element or at least one connection element in the feedthrough or the plug part (in particular in a hermetically sealed embodiment) consists at least in part of a shape-memory alloy.

A feedthrough or a plug part is thus provided of which the connection element(s) is/are insensitive to position deviations in the production process and which is/are insensitive to bending or other types of deformation. Furthermore, a process is provided which “heals” the feedthroughs or plug parts having bent or deformed connection or contact elements so that these lie again within the predefined tolerance ranges and can be used without finishing steps at the time of final assembly of the device or the modular unit.

One or more of the advantages specified below can be achieved with the present invention, at least in expedient embodiments:

Process steps for the supplier and client can be spared or consolidated.

Reduction of rejection as a result of thermal post-treatment process.

Improved demolding properties of the feedthroughs from the manufacturing aids as a result of selective relaxation of the feedthrough.

In one embodiment of the present invention the connection element or at least one connection element consists at least in part of a shape-memory alloy demonstrating a one-way shape-memory effect. In an alternative embodiment, or in a further embodiment which can also be combined with the aforementioned embodiment, the connection element or at least one connection element consists at least in part of a shape-memory alloy demonstrating two-way shape-memory effect.

In a further embodiment of the present invention, provision is made so that the connection element or at least one connection element is joined from at least two parts and at least one of the parts consists solely of a shape-memory alloy. In one embodiment, the connection element or at least one connection element has an outer tube and a core, and the core consists of a shape-memory alloy. Provision can also be made so that the connection element or at least one connection element consisting only of a shape-memory alloy has a thin coating, or so that the portion of the, or a connection element consisting of a shape-memory alloy has a thin coating.

In a further embodiment, the shape-memory alloy or at least one shape-memory alloy has super-elastic properties in order to form a connection element or part thereof.

Besides the actual connection or contact elements, the inventive concept can also be applied to the ground connection or grounding pin of an electro-medical device or a modular unit. The feedthrough or the plug part therefore has a grounding pin which is formed at least in part of a shape-memory alloy, in particular one that demonstrates super-elastic behavior.

The proposed improvement relates ultimately to a medical electronic device, in particular formed as a cardiac pacemaker, implantable cardioverter or cochlear implant, or a medical electronic modular unit, in particular formed as an implantable electrode line.

The method according to the present invention is characterized in that the finished, assembled feedthrough or the assembled plug part is heated, prior to the assembly of the device or the modular unit, to a temperature above the characteristic phase-transition temperature, in particular in a climatic chamber or by resistance heating or via thermal conduction from an applied heating element. With regard to the above-mentioned ground connection, a grounding pin in thermal contact with the feedthrough flange (or a corresponding plug sleeve or a plug flange) can be heated, in a specific procedure, by inductive heating of the feedthrough flange.

Further embodiments, features, aspects, objects, advantages, and possible applications of the present invention could be learned from the following description, in combination with the Figures, and the appended claims.

### DESCRIPTION OF THE DRAWINGS

Advantages and expedient features of the present invention will become clear incidentally from the description of exemplary embodiments with reference to the drawings, in which:

FIG. 1 shows a schematic, partly cut-away illustration of an implantable medical electronic device,

FIG. 2 shows a schematic illustration (sectional view) of a feedthrough flange of conventional design,

FIGS. 3A-3C show sketched illustrations in order to explain a first variant of the present invention,

FIGS. 4A-4D show sketched illustrations in order to explain a second variant of the present invention,

FIG. 5 shows a schematic perspective view of an embodiment of the plug part according to the present invention, and

FIG. 6 shows a schematic longitudinal sectional view of an embodiment of a feedthrough according to the present invention.

### DETAILED DESCRIPTION

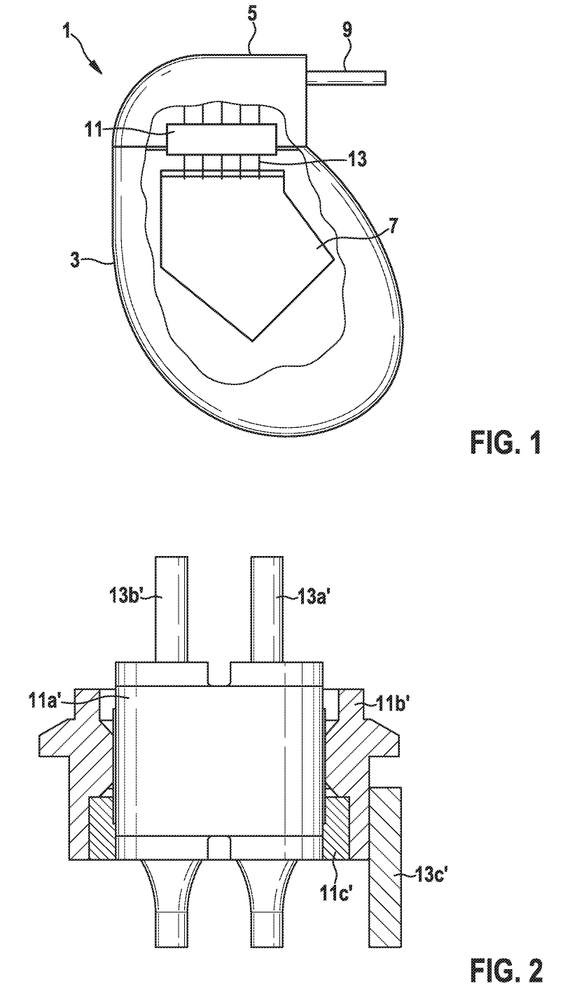

FIG. 1 shows a cardiac pacemaker 1 having a pacemaker housing 3 and a head part (header) 5, in the interior of which there is arranged a printed circuit board (PCB) 7 in addition to other electronic components, there also being an electrode line 9 connected to the line connection (not shown) arranged in the header 5 of said pacemaker 1. A feedthrough 11 provided between the device housing 3 and header 5 comprises a multiplicity of connection pins 13. The connection pins 13 are plugged at one end through a corresponding bore in the printed circuit board 7 and are soft-soldered thereto. The soldering can be performed at a soldering temperature of 230° C., for example.

FIG. 2 shows, in a sectional illustration along a central plane of section, a feedthrough 11′ of conventional design, which comprises a ceramic insulating body 11a' and a feedthrough flange 11b′ milled from solid material, which surrounds the insulating body 11a′. A solder ring 11c′ is placed in a recess, annularly surrounding the insulating body 11a′, at the lower end of the feedthrough flange 11b′; the insulating body 11a′ is connected there to the feedthrough flange in a hermetically sealed manner by means of a hard-soldering method. Long and short connection pins 13a′, 13b′ penetrate through the insulating body 11a′, and a grounding pin 13c′ is welded on outside to the feedthrough flange 15′. A peripheral flange edge at the feedthrough flange 15′ serves as a welding edge when the flange is inserted into a clearance or bore of a device housing (not shown) and is welded there.

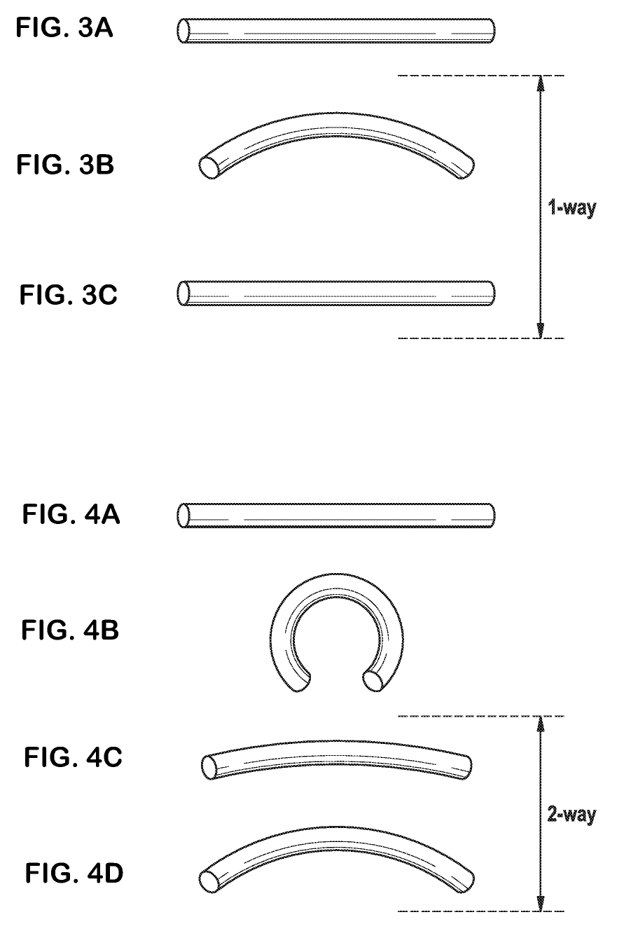

FIGS. 3A-3C and 4A-4D each show, in a sketched manner, various states of a cylindrical pin serving as connection element and made of a shape-memory alloy (for example NiTi or nitinol, NiTiCu, CuZnAl, CuAlNi, FeMnSi, FeNiCoTi), which can be used in a feedthrough or a plug part designed in accordance with the present invention. The illustrations serves to show the form or dimensional changes and mechanical behavior of said pin, irrespective of the specific installation situation in a feedthrough or a plug part and without consideration of influences of the installation situation on the dimensional changes and mechanical behavior.

In FIG. 3A, the pin is in the delivered state and is processed for a feedthrough. During the joining process, the originally set temperature of the shape-memory wire is shifted upwardly by a few degrees. As symbolized in FIG. 3B, the soldered pin may be damaged on account of bending or deformation. The feedthrough with pin will be classed as a rejection at the time of inspection of the observance of position/form tolerances, since the contact element does not meet the specifications and cannot be reliably connected to the contacts in the subsequent processes.

As symbolized in FIG. 3C, the deformed pin can be returned to its original form by heating by use of the one-way shape-memory effect, and therefore the position of its end to be connected lying within the tolerance range can be re-established. The heat treatment is performed in a convection oven or in a climatic chamber. It is advantageous to couple the heat treatment with a subsequent process (heat treatment by pre-heating in a reflow process, plasma cleaning or plasma activation prior to the further processing).

FIG. 4A also shows the pin in the delivered state, as it can be processed for a feedthrough or a plug part. The modified form/end face position of the pin is trained into the material in accordance with FIG. 4B (heat treatment). The pin is then deformed for assembly and is inserted into the feedthrough or the plug part in the state shown in FIG. 4C. A joining process is then performed, for example, at approximately 800° C. During the joining, the originally set temperature of the shape-memory wire is shifted upwardly by a few degrees.

As a result of the two-way shape-memory effect, the pin transfers, as it cools, into the defined form/position previously trained. The trained dimensional change can be used repeatedly to retrieve pins from equipping devices. Once demolded, the pins are transferred into their end form by means of a heat-treatment process.

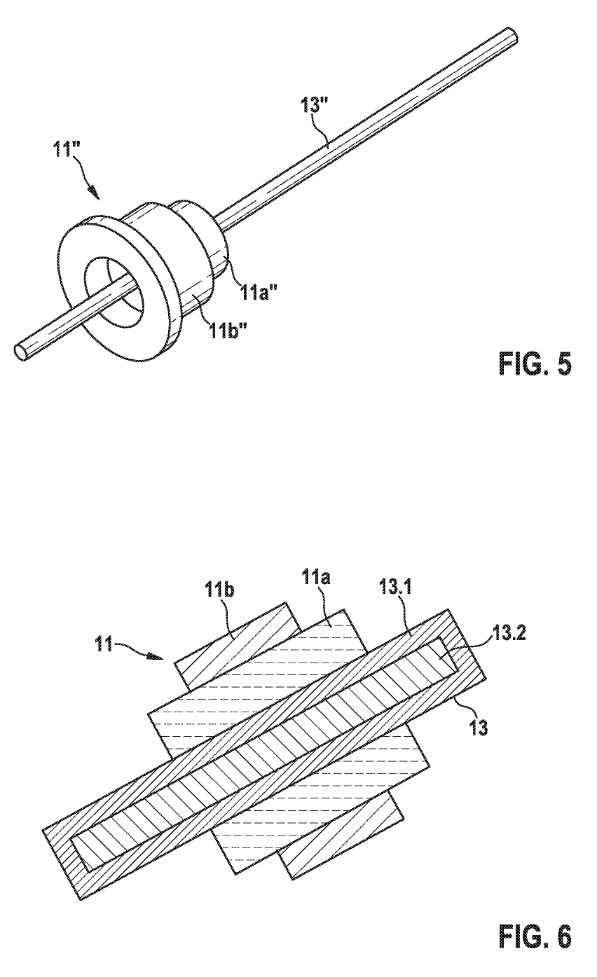

FIG. 5 shows a perspective view of a plug part 11″, for example, as a component of an electrode line, which comprises an insulating body 11a″, a plug flange 11b″ surrounding the insulating body, and a connection pin 13″ penetrating through the insulating body centrally and made of a shape-memory alloy.

FIG. 6, in a schematic longitudinal sectional illustration, shows a feedthrough 11 which comprises an insulating body 11a, a feedthrough flange 11b surrounding the insulating body 11a, and a connection pin 13 penetrating through the insulating body 11a centrally. The connection pin 13 is constructed in two parts from a core 13.1 made of a shape-memory alloy and an outer tube 13.2 made of a conventional conductive metal.

The present invention can also be carried out in a large number of modifications of the examples presented here and aspects of the present invention detailed further above.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof.

Additionally, the disclosure of a range of values is a disclosure of every numerical value within that range.

I/We claim:

1. A feedthrough of a medical electronic device, which is implantable and has a device housing in which electronic and/or electrical function units are housed and which has a housing opening closed by the feedthrough, wherein the feedthrough has an insulating body, a feedthrough flange surrounding the insulating body and fixed to the housing opening, and at least one connection element penetrating through the insulating body for the external connection of at least one component of the device,
wherein the connection element or at least one connection element consists at least in part, in particular substantially fully, of a shape-memory alloy.

2. The feedthrough according to claim 1, wherein the connection element or at least one connection element consists at least in part of a shape-memory alloy demonstrating a one-way shape-memory effect.

3. The feedthrough according to claim 1, wherein the connection element or at least one connection element consists at least in part of a shape-memory alloy demonstrating a two-way shape-memory effect.

4. The feedthrough according to claim 1, wherein the connection element or at least one connection element is joined from at least two parts and at least one of the parts consists fully of a shape-memory alloy.

5. The feedthrough according to claim 4, wherein the connection element or at least one connection element has an outer tube and a core, and the core consists of a shape-memory alloy.

6. The feedthrough according to claim 1, wherein the connection element or at least one connection element consisting fully of a shape-memory alloy has a thin coating, or a portion of the connection element or at least one connection element consisting of a shape-memory alloy has a thin coating.

7. The feedthrough according to claim 1, wherein the shape-memory alloy or at least one shape-memory alloy has super-elastic properties in order to form a connection element or part thereof.

8. The feedthrough according to claim 1, which has a grounding pin, which is formed at least in part from a shape-memory alloy, in particular one that demonstrates super-elastic behavior.

9. A medical electronic device having a feedthrough according to claim 1, in particular formed as a cardiac pacemaker, implantable cardioverter or cochlear implant.

10. A method for producing a device according to claim 9, wherein the finished, assembled feedthrough is heated prior to the assembly of the device to a temperature above the characteristic phase-transition temperature of the shape-memory alloy, in particular in a climatic chamber or by resistance heating or via thermal conduction from an applied heating element.

11. The method according to claim 10, wherein a grounding pin in thermal contact with the feedthrough flange is heated by inductive heating of the feedthrough flange.

12. A plug part of a medical electronic modular unit, which has an insulating body and at least one connection element penetrating through the insulating body for the external connection of an electrical line of the modular unit, wherein the connection element or at least one connection element consists at least in part, in particular substantially fully, of a shape-memory alloy.

13. The plug part according to claim 12, wherein the connection element or at least one connection element consists at least in part of a shape-memory alloy demonstrating a one-way shape-memory effect or demonstrating a two-way shape-memory effect, and/or the connection element or at least one connection element is joined from at least two parts and at least one of the parts consists fully of a shape-memory alloy.

14. A medical electronic modular unit having a plug part according to claim 12, in particular formed as an implantable electrode line.

15. A method for producing a modular unit according to claim 14, wherein the finished plug part is heated prior to the assembly of the modular unit to a temperature above the characteristic phase-transition temperature of the shape-memory alloy, in particular in a climatic chamber or by resistance heating or via thermal conduction from an applied heating element.
